SEQUENCE LISTING (8 sequences)

SEQ ID NO: 1 - skipped

SEQ ID NO: 2 - DNA, 58 bp, synthetic construct
  source 1..58: mol_type = other DNA
acttgacagtctggtctccgatgacgtctcagatgagcaaaggagaagaacttttcac

SEQ ID NO: 3 - DNA, 54 bp, synthetic construct
  source 1..54: mol_type = other DNA
atgcagcattggtctctcttatcgtctcacttatttgtagagctcatccatgcc

SEQ ID NO: 4 - DNA, 24 bp, synthetic construct
  source 1..24: mol_type = other DNA
caaaaaagggaataagggcgacac

SEQ ID NO: 5 - DNA, 21 bp, synthetic construct
  source 1..21: mol_type = other DNA
gttagctcactcattaggcac

SEQ ID NO: 6 - DNA, 20 bp, synthetic construct
  source 1..20: mol_type = other DNA
gaataagggcgacactgttg

SEQ ID NO: 7 - DNA, 46 bp, synthetic construct
  source 1..46: mol_type = other DNA
acttgacagtctggtctccgatgagcaaaggagaagaacttttcac

SEQ ID NO: 8 - DNA, 44 bp, synthetic construct
  source 1..44: mol_type = other DNA
gtatgcagcattggtctctcttatttgtagagctcatccatgcc

(19) United States
(12) Patent Application Publication
(10) Pub. No.: US 2024/0344052 A1
(43) Pub. Date: Oct. 17, 2024

(54) ERROR REDUCTION IN GENE SYNTHESIS

(71) Applicants: Hal S. Padgett, Vacaville, CA (US); Matthew McGee, Vacaville, CA (US); Kerstien Padgett, Vacaville, CA (US)

(72) Inventors: Hal S. Padgett, Vacaville, CA (US); Matthew McGee, Vacaville, CA (US); Kerstien Padgett, Vacaville, CA (US)

(21) Appl. No.: 18/430,419

(22) Filed: Feb. 1, 2024

Related U.S. Application Data
(60) Provisional application No. 63/582,122, filed on Sep. 12, 2023.
(60) Provisional application No. 63/451,420, filed on Mar. 10, 2023.
(60) Provisional application No. 63/443,977, filed on Feb. 7, 2023.

(51) Int. Cl. C12N 15/10; C12N 15/70; C12Q 1/02; C12Q 1/6806

(52) U.S. Cl. CPC C12N 15/1003; C12N 15/1096; C12N 15/70; C12Q 1/02; C12Q 1/6806

(57) ABSTRACT

Presented here are methods for identifying or enriching for error-free DNA sequences occurring within an initial population of DNA molecules, wherein at least a plurality of the initial population of DNA molecules contains a desired error-free sequence.

### CROSS REFERENCE TO RELATED APPLICATIONS

This application claims priority from U.S. Provisional applications 63/443,977 filed 7 Feb. 2023, 63/451,420 filed 10 Mar. 2023, and 63/582,122 filed 30 Sep. 2023.

### REFERENCE TO THE SEQUENCE LISTING

The present application is being filed along with a Sequence Listing in electronic format. The Sequence Listing file entitled NBT1016US1.xml was created on Jan. 31, 2024 and is 7,427 bytes in size. The information in electronic format of the Sequence Listing is incorporated herein by reference in its entirety.

### BACKGROUND OF THE INVENTION

The ability to quickly obtain and modify genes for use in recombinant systems is a crucial aspect of biotechnology research. Synthesis of genes to precise sequence specifications has become more common as an alternative to isolation of genes from their native sources. In addition to producing single genes for analysis, recent trends in cost and production capacity are making it increasingly economical to produce DNA for rapid prototyping, optimization studies, and data storage applications.

In recent years, several companies have specialized in integrating and streamlining the steps involved in DNA synthesis to produce customized genes to order. These companies compete based on both cost and turnaround time. Typical timeframes from receipt of orders to shipment of the final synthetic products to the customer range from about a week for genes less than a kilobase in length to several weeks for longer sequences.

The process of gene synthesis is typically performed by annealing a series of overlapping oligonucleotides followed either by extension with a DNA polymerase to create double stranded products in the case of partially overlapping oligonucleotides, or by ligation of fully overlapping oligonucleotides. Through additional cycles of melting, annealing and extension, the sizes of those products increase until the full-length gene is assembled. The assembled product is then amplified by polymerase chain reaction (PCR) using terminal primers to generate the final DNA for cloning and sequence analysis.

One barrier to reducing the cost and turnaround time for gene synthesis is the occurrence of errors in the synthetic DNA. Most of these errors arise from misincorporation events during the synthesis of the oligonucleotides. Their distribution within a given oligonucleotide appears to be largely random, although some oligonucleotides may contain more errors than others, probably as a result of conditions of the synthesis or influences from the DNA sequence.

Fractionation of oligonucleotides to enrich for those that have fewer errors can be performed using polyacrylamide gel electrophoresis (PAGE) or high-performance liquid chromatography (HPLC). These approaches can be effective but can also be time consuming and expensive. Other approaches to reducing the frequency of errors in the pool of oligonucleotides include removal by binding or degradation of mismatched DNAs after duplex formation with a complementary strand. However, these approaches can be laborious and require large initial quantities of oligonucleotide to ensure an adequate supply for subsequent manipulation.

Errors in gene synthesis can also be reduced after assembly of the gene from the set of oligonucleotides. Because of the random distribution of errors, a given error-containing strand within the population is unlikely to contain the same errors at the same positions in any given complementary strand. Therefore, base mismatches should result at the sites of errors when individual strands are dissociated from one another (melted) and then allowed to reanneal randomly with complementary strands in the population. Those mismatches can be bound and removed by DNA mismatch binding proteins or cleaved with mismatch specific endonucleases to render them less than full-length. In either case, the remaining full-length strands should contain fewer mismatches, and thus, would be expected to carry fewer errors. In 2009, a product and accompanying method for DNA error reduction was introduced that combined enzymatic cleavage of DNAs at mismatches, with subsequent PCR re-assembly of the resulting fragments to reconstitute full-length error-reduced DNA products (Carlson R. The changing economics of DNA synthesis. Nature Biotechnology 27:1091-1094 (2009)). This became a standard approach to reduction of DNA errors in synthetic genes produced in industrial workflows as well as by individual researchers (Kosuri S, Eroshenko N, LeProust E M, Super M, Way J, Li J B, Church G M. Scalable gene synthesis by selective amplification of DNA pools from high-fidelity microchips. Nature Biotechnology 28:1295-1299 (2010); Dormitzer P R, Suphaphiphat P, Gibson D G, J Craig Venter. Synthetic generation of influenza vaccine viruses for rapid response to pandemics. Science Translational Medicine 5:185ra68 (2013).

Another way to improve chances for identifying error free genes is to sequence a greater number of candidates. The random distribution of errors means that error-free genes will occur at a frequency that varies primarily as a function of error rate and gene length. The longer the synthesized gene, the greater the likelihood a given candidate will contain at least one error, and the larger the number of candidates that must be sequenced. Prices per unit length of synthetic DNA are generally higher for longer DNAs than shorter ones, in great part because of increased sequencing costs. Turnaround times can also be longer because of the higher likelihood of errors in longer sequences and because longer sequences may require assembly of multiple sequence reads.

Recent advances in molecular biology, such as biochemical pathway engineering, have increased the demand for longer synthetic DNAs as well as for producing sets of gene variants for optimization studies. The need for longer synthetic genes containing fewer errors is increasingly accompanied by pressures for reduced costs and faster turnaround. Therefore, a need exists for synthetic DNA error reduction methods that do not add appreciably to the cost or time requirements of the gene synthesis workflow.

Presented here are methods for identifying or enriching for error-free DNA sequences occurring within an initial population of DNA molecules, wherein at least a plurality of the initial population of DNA molecules contains a desired error-free sequence.

### BRIEF SUMMARY OF THE INVENTION

Here, we present methods for reducing errors in synthetic genes that permit the use of unpurified oligonucleotides, requires little time to complete, and can fit into a variety of widely practiced gene synthesis and cloning workflows that may optionally also include additional error reduction steps. Because error rates in the synthetic DNA products can be dramatically reduced, less sequencing may be required to identify error-free genes. Also because of the reduced error rates, longer error-free genes can be synthesized. For typical genes of around 1 kilobase, this method can provide benefits in both the cost and time of synthesis. For longer DNAs, and for multifragment assemblies of synthetic DNAs, however, the benefit can be compounded by the impact of the reduced sequencing burden for producing long stretches of error-free DNA. Even if perfectly error-free clonal synthetic DNAs are not required, the methods herein can be employed to reduce errors in populations of non-clonal (pooled) synthetic DNAs. Moreover, these methods can apply to all forms of DNA, including DNAs in which non-native nucleotide base-pairs are incorporated to expand the DNA alphabet and otherwise extend the information-coding capacity of the DNA product. Similarly, these methods apply not only to synthetic DNAs, but also to DNA products derived from such processes as reverse transcription and PCR, which are known to introduce mutations into DNA.

These methods each rely upon the known mechanism of DNA “co-repair” in which DNA structures that, on their own, are not efficiently recognized by a cellular DNA mismatch repair system are eliminated in the process of repairing portions of the same DNA molecule containing other, recognized mismatch structures. DNA structures that are recognized by the DNA mismatch repair system include DNA base substitutions and small insertions and deletions of fewer than about three or four nucleotides, such as are typically found to be fairly randomly distributed within a population of synthetic DNA fragments. In contrast, DNA loop structures that result from insertions or deletions of larger sequences of about five or more nucleotides in one strand of the DNA relative to the other strand are not generally recognized as a trigger for the mismatch repair process.

One embodiment of the invention comprises an in vitro method of isolating an unmutated DNA sequence of interest comprising: preparing a linear hemi-methylated double stranded plasmid vector DNA wherein the unmethylated strand (strand A) encodes a functional selectable marker gene and the methylated strand (strand B) has one or more insertions and/or deletions of four or more bases within the selectable marker gene of strand B relative to strand A and wherein the plasmid vector encodes a screenable marker gene; preparing a pool of double stranded DNAs containing a DNA sequence of interest using PCR overlap assembly from a pool of overlapping oligonucleotides wherein some of the resulting double stranded DNAs contain mutations, including insertions and deletions of various sizes, as well as base substitutions; denaturing and reannealing the double stranded DNAs to form heteroduplexes and homoduplexes; ligating the heteroduplexes and homoduplexes with the linear hemi-methylated double stranded plasmid vector to generate circular plasmid molecules; transforming the circular plasmid molecules into host cells having a DNA repair system; allowing time for the host cells to form colonies; scoring the colonies for the level of expression of the screenable marker; choosing colonies with a discernibly different level of signal from the screenable marker than the level of signal observed with a control construct; isolating plasmid DNA from the chosen colonies; and evaluating the DNA sequence of the plasmids from the chosen colonies to detect plasmids with an unmutated DNA sequence of interest.

### BRIEF DESCRIPTION OF THE DRAWINGS

FIG. 1 shows a schematic that depicts the reciprocal hemimethylated looped-heteroduplex test vector molecules produced from non-methylated pMM12 and methylated pMM13.

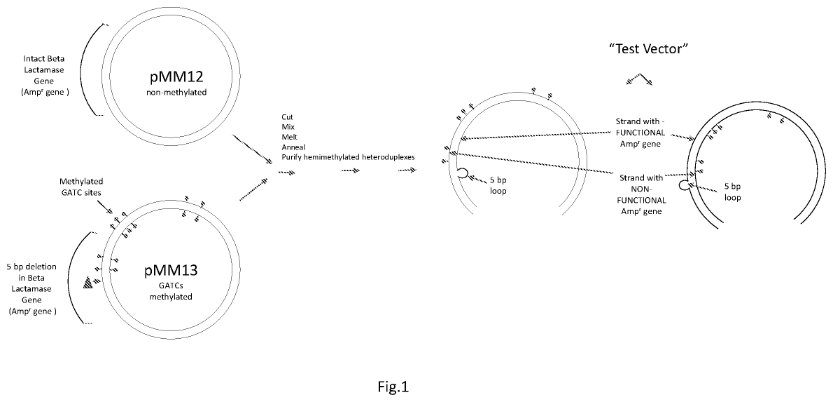

FIG. 2 shows insertion of double-stranded DNA inserts from a population of heteroduplexes and homoduplexes into test vector. Insert at left contains a mutation in one of the strands, causing a mismatch. Insert at right depicts a homoduplex.

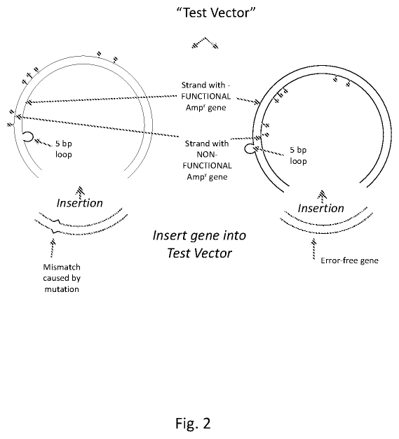

FIG. 3 shows the expected consequences of transformation of each of the depicted circular test vector/insert molecules, as depicted in FIG. 2, into E. coli. The molecule on the left containing a mismatch in the insert sequence engages with the methyl-directed DNA mismatch repair system, ultimately leading to co-repair of the loop in the beta lactamase gene and its conversion to a double-stranded DNA encoding a dysfunctional beta lactamase, thereby preventing cells containing test vector molecules repaired in this way from growing on selective media. If “wrong-strand” co-repair occurs, then the resulting functional beta lactamase can support bacterial growth, thereby contributing to higher background levels of insert sequences more likely to contain mutations. The molecule on the right containing a homoduplex insert (no mismatch) is not expected to engage with the DNA mismatch repair system. Subsequent replication of each of the two strands of that input test vector results in two plasmids, differing only in the beta lactamase gene (wild type functional and 5 base pair deleted), that replicate in mixed form in those cells and at least a subset of the cells they give rise to.

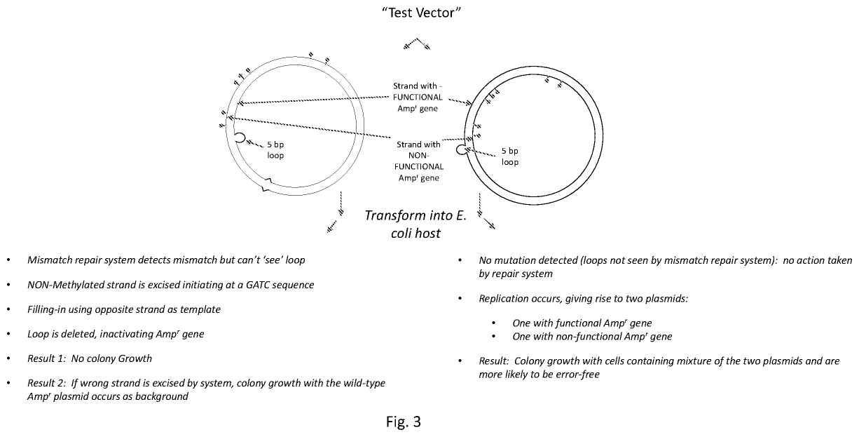

FIG. 4 shows the observed fluorescence from colonies arising from test vector constructs containing inserts with mismatches (left) or inserts without mismatches (right). Fluorescence levels from colonies on the left are as expected and are similar in intensity to the controls in which inserts are cloned into pMM12-M, a non-loop-containing vector in which both strands encode a functional beta lactamase. On the right, colonies arising from non-mismatched insert DNAs, and thereby supporting co-replication of functional/nonfunctional beta lactamase plasmids, exhibit a discernably higher level of fluorescence than on the left or in the non-loop vector control. The observed intensification of the GFP signal from such colonies is unexpected, especially since the GFP gene itself and its promoter are identical in both strands, and thus would not have been expected to bring about fluorescence levels any higher than either the colonies on the left side of the figure or the control colonies.

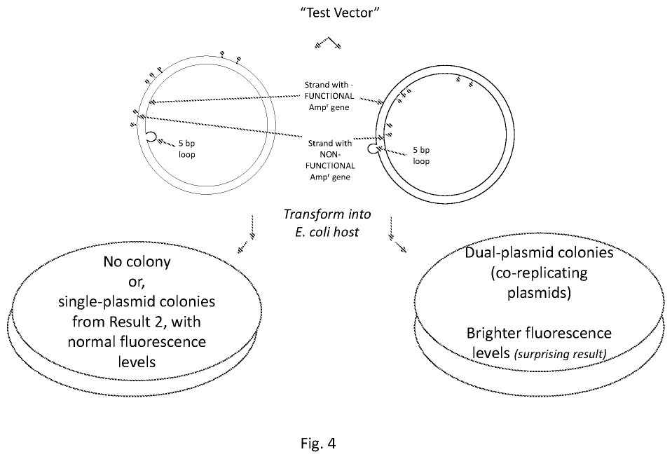

### DETAILED DESCRIPTION OF THE INVENTION

The following detailed description is of the best currently contemplated modes of carrying out exemplary embodiments of the invention. The description is not to be taken in a limiting sense but is made merely for the purpose of illustrating the general principles of the invention, since the scope of the invention is best defined by the appended claims.

Broadly, an embodiment of the present invention generally is a method for reducing errors in synthetic DNAs.

Here, we present a method for reducing errors in synthetic DNAs that permits the use of unpurified oligonucleotides, requires little time to complete, and can fit into a variety of widely practiced gene synthesis workflows. Because error rates in the synthetic products can be dramatically reduced, less sequencing may be required to identify error free genes. Moreover, because of the reduced error rates, longer genes can be synthesized. For typical DNAs of around 1 kilobase, this method can provide benefits in both the cost and time of synthesis. For longer DNAs, however, the benefit can be compounded by the impact of the reduced sequencing burden for synthesizing long stretches of error-free DNA.

DNA errors are a significant limitation to the fields of molecular biology and synthetic biology, particularly in synthetic gene synthesis, DNA data storage, and other technologies that depend on error-free or error-reduced DNAs. These errors can take the form of insertions and deletions of one or more nucleotides (indels) or substitution of the desired nucleotide for a different nucleotide. Depending on the application, just a single indel or substitution error in a DNA molecule may be unacceptable. The rate of errors in DNAs will depend on the technologies employed but will ultimately influence the maximum length of error-free DNA that can be reliably attained. Therefore, technologies that reduce errors in DNAs are needed. Although some technologies such as the ErrASE™-mediated enzymatic DNA mismatch cleavage and reassembly process (Lubock NB, Zhang D, Sidore AM, Church GM, Kosuri S. A systematic comparison of error correction enzymes by next-generation sequencing. Nucleic Acids Res 45:9206-9217 (2017)) already exist that can at least partially mitigate this problem, error rates in DNA are often still unacceptably high for many applications.

Presented here are methods for identifying or enriching for error-free DNA sequences occurring within an initial population of DNA molecules, wherein at least a plurality of the initial population of DNA molecules contain a desired sequence. These methods each rely upon the known mechanism of DNA “co-repair” in which DNA structures that are not efficiently recognized by a cellular DNA mismatch repair system are eliminated in the process of repairing portions of the same DNA molecule containing other, well-recognized mismatch structures. DNA structures that are well-recognized by the DNA mismatch repair system include DNA base substitutions and small insertions and deletions of fewer than about three or four nucleotides, such as are typically found to be fairly randomly distributed within a population of synthetic DNA fragments. In contrast, DNA loop structures that result from insertions or deletions of larger sequences of about five or more nucleotides in one strand of the DNA relative to the other strand are known to be much less efficiently recognized by the DNA mismatch repair system of E. coli, and thus do not trigger the mismatch repair process.

In E. coli, activation of the methyl-directed DNA mismatch repair system by detection of a DNA mismatch leads to strand nicking at a hemimethylated GATC sequence situated in rough proximity to the DNA mismatch, followed by excision of long stretches from the nicked strand. In a plasmid DNA, typical excision tracts are on the order of thousands of bases, long enough to extend across a large proportion, if not the entirety, of the plasmid DNA. Because repair events typically cover such long stretches of sequence, if a loop of five nucleotides or greater (unrecognized loop) is present in the DNA molecule undergoing a repair event that was triggered by a mismatch located elsewhere on the molecule, there is a strong likelihood that both sites will be included in the repair tract. Inclusion of the unrecognized loop in the repair tract ultimately results in its being resolved by excision and resynthesis of one strand or the other, eliminating the loop structure, and thereby bringing about its co-repair along with the DNA mismatch structure that originally triggered the correction event. The resulting fully base paired, double-stranded DNA matches the template strand that had not been excised.

In the examples cited herein, the loop is situated in an antibiotic resistance gene of a plasmid vector backbone. The double-stranded DNA vector containing this loop structure was created by hybridizing two different DNA molecules with one another, with one strand encoding a wild-type beta-lactamase gene and the other strand encoding a beta-lactamase gene made dysfunctional by the introduction of a five-nucleotide deletion into the coding region of the gene. Further, the vector DNA is hemimethylated, in that methyl groups are present on the adenine of GATC sites of the strand encoding the dysfunctional beta lactamase, while the GATC sequences of the other strand that encodes the wild-type beta lactamase gene remain unmethylated. This vector is termed the “test vector” herein.

To create the DNA fragment to be inserted into the test vector, a population of synthetic DNA fragments is melted and reannealed to form either homoduplex DNA molecules in which the strands are perfectly base-paired, or heteroduplex DNA molecules in which imperfectly base-paired strands are formed by mutations in one or both strands. Once inserted into the hemimethylated test vector and transformed into E. coli, mismatches present in the inserted sequences are detected by the cellular methyl-directed DNA mismatch repair system, which is then triggered to preferentially excise large sections of the unmethylated strand, followed by its resynthesis using the methylated strand as the reference template for polymerization. In the test vector system described here, expected repair events by this system would bring about co-repair of the loop structure in the beta lactamase gene such that the resulting double-stranded DNA encodes a non-functional beta lactamase gene. This plasmid would be incapable of supporting bacterial growth on selective medium, so molecules co-repaired in this way would essentially be deleted from the population.

If no mutations are present in the melted and reannealed DNA fragment that is inserted into the hemimethylated loop-containing test vector, then DNA repair is not expected to occur. In turn, if no repair occurs on such molecules prior to plasmid replication in the host, then the co-replication of two independent plasmids is likely to result. The plasmid arising from one strand will encode the functional beta lactamase gene, and the plasmid arising from the other strand will contain a nonfunctional (five base pair deletion) beta lactamase gene. We found that replication of this latter deletion plasmid does indeed occur, and that it co-replicates persistently with the functional beta lactamase plasmid in the E. coli, ultimately (and surprisingly) representing approximately 75%-90% of the plasmid material recovered from such cultures. Essentially, the occurrence of colonies that contain the co-replicating plasmids indicates that error correction had most likely not occurred on the initial plasmid molecule that was transformed into the E. coli cell that eventually became a colony on solid media. Lack of error correction on the molecule can be taken as an indication that the inserted DNA fragment was likely to be free of detectable errors creating DNA mismatches that are recognizable to the methyl-directed DNA mismatch repair system in the first place. Thus, in this system, mixed-plasmid colonies are likely to contain fewer errors on average in the inserted DNA fragments than in the initial population of DNA fragments.

Co-repair events occurring with the wrong directionality with respect to the methylation signals in the hemimethylated test vector DNA would result in both strands containing a functional beta lactamase gene and would thus be capable of conferring antibiotic resistance and growth of the bacterial host in the presence of selective media. In the context of the error reduction systems presented here, such ‘wrong-strand’ repair events represent a background of undesirable sequences considered more likely to contain errors than sequences in plasmids that had not undergone DNA repair occurring in cells that contain a mixture of the co-replicating plasmids derived from the original strands of the test vector. It is desirable to avoid recovery of insert sequences from this background category of wrong-strand repair products.

To make practical use of the insight that co-replicating plasmids suggest the presence of error-free insert sequences, colonies would either need to be tested individually for the presence of the plasmid with the dysfunctional beta lactamase gene (an impractical process at best), or a second loop would need to be included in the vector construct along with another gene that would serve as a phenotypically scorable marker (as taught by U.S. Pat. No. 6,709,827). Cells with such a marker would be scorable for the presence or absence of the marker gene function, or in the case of co-replicating plasmids in mixed form, for the intermediate state of the two, as described by Faham et al. (PMID: 8808468, PMID: 11487569, U.S. Pat. No. 6,709,827). However, an additional loop would create additional complexity in the construction of the vector. What is needed in the art is a way to employ a single-loop co-repair vector such as the one described here to phenotypically distinguish colonies containing the two plasmids derived from each of the original input strands of the test vector versus those colonies containing only a single plasmid.

In the course of experiments to characterize the single-loop co-repair vector described herein, it was observed that when a gene encoding a fluorescent reporter protein (green fluorescent protein, GFP) was present elsewhere in the vector, bacterial colonies supporting co-replicating plasmids as described above emitted discernably higher levels of fluorescence than background colonies containing only a single plasmid which had presumably resulted from DNA error correction occurring on the methylated (wrong) strand of the introduced test vector. These brighter, mixed-plasmid colonies were also discernably brighter than colonies arising from cells containing the identical control vector containing only the wild-type version of the beta lactamase gene. This result was unexpected in that the intensification of the GFP signal was not caused by any change in the fluorescent marker gene itself, or in its promoter sequences, so no differences in its signal output would have been expected. Moreover, if following the teachings of Faham et al. (PMID: 8808468, PMID: 11487569, U.S. Pat. No. 6,709,827) it would have been expected that sequence differences of some kind would need to be present between the strands of the marker gene to effect changes in its signal output, and that such signal might be expected from those teachings to be intermediate between the two phenotypic states associated with plasmid having only wild-type marker or plasmid having only mutant marker. The result presented here is important in that it provides a practical and effective means to discriminate against background colonies that may have undergone DNA repair on the incorrect strand (wrong-strand repair), and therefore, to improve the identification of colonies supporting co-replicating plasmids, which, as is presented herein, are far more likely to contain error free insert sequences.

Another embodiment is presented here in which reduced-error sequences are recovered from individual clonal populations such as bacterial colonies, or from pooled populations of bacterial cells such as in liquid cultures inoculated with bacterial transformation reactions. In this embodiment, insert DNA sequences are recovered preferentially from plasmids encoding the deletion-containing version of the beta-lactamase gene, while avoiding the recovery of insert sequences present in the plasmids encoding the wild-type version of the beta lactamase gene. In essence, only cells supporting co-replication of the two plasmids derived from each strand of the input test vector (and thus only the cells more likely to contain error-free insert sequences) are expected to contain the form of plasmid with the dysfunctional beta lactamase gene.

Both plasmids in such co-replicating plasmid populations are expected in most cases to contain identical sequences in their entirety, except for the modification that was introduced into the test vector to create the co-repair target loop. In the examples presented here, the modification was a five basepair deletion in the beta lactamase gene. To ensure selective recovery only from plasmids containing this deletion, PCR amplification could be performed in which one of the primers is designed so that it can only successfully prime DNA synthesis from the plasmids encoding the dysfunctional beta lactamase. This PCR amplification can be performed using template DNA derived either from clonal isolates like bacterial colonies, or from non-clonal, or pooled, samples such as in liquid culture of bacterial transformation reactions. To simplify the design and use of oligonucleotide primers for this PCR, it can be worthwhile for the loop-containing test vector to be constructed so that the strand encoding the dysfunctional selectable marker gene has been modified by a sequence insertion instead of by a deletion, as was used in the examples described herein. Such an insertion can represent target sequences for successful PCR primer binding that will be substantially or entirely absent in the other strand encoding the functional selectable marker.

Plasmids encoding the desired population of insert sequences can also be recovered by selective inactivation of the population of plasmids containing the functional beta lactamase gene with a restriction endonuclease site present at the loop site, but only in the test vector strand encoding the functional beta lactamase. The site will be absent in the strand of the test vector encoding the dysfunctional beta lactamase. Ideally, both strands of the test vector would also encode a fully functional resistance gene to a second antibiotic. Plasmids from transformed cells cultured under selection with a beta lactam antibiotic are treated with the endonuclease and the resulting mixture of linearized and still-circular plasmids is transformed into E. coli and grown in the presence of the second antibiotic. As transformation with circular DNA is far more efficient than with linear DNA, the resulting transformed cells will predominantly harbor plasmids originating from the test vector strand containing the dysfunctional beta lactamase but not the restriction site.

While these methods represent practical approaches to selective recovery of error-free sequences from the larger population of plasmid sequences, we envision this invention to cover any method capable of allowing selective recovery of insert sequences contained in plasmids with the dysfunctional selective marker gene, or stated differently, any method capable of selectively avoiding recovery of insert genes contained in plasmids with the functional selectable marker gene, especially those arising from earlier repair events on the input test vector. Additionally, the examples presented here contemplate a single loop structure for co-repair, but any number of such loops can be used as might be practical. Such loops may be present in the same or additional selectable marker genes (for either positive or negative selection), in scoreable genes such as those encoding fluorescent proteins like GFP or enzymes enabling a colorimetric readout, or the loop or loops may be present in sequences not associated with a phenotype, but that still allow plasmid DNA containing the dysfunctional beta lactamase (in this instance) to be selectively addressed by any means, including by the use of a selective PCR primer(s) and/or cleavage by restriction endonuclease.

This method is particularly well-suited for automated workflows, in that clonal isolation of individual DNAs is not required to obtain effective levels of error reduction. Reduced-error, synthetic insert DNAs recovered in this manner can then be used directly or taken through another round of error reduction, such as by introduction into the loop vector described above, that permits enrichment for error-free DNAs via the use of the fluorescence intensification mechanism described herein, or by other methods such as the ErrASET process. Likewise, other means of error reduction can also be used in steps prior to this method.

We believe that this embodiment is a significant improvement upon the art as most relevantly reflected in the teachings of Faham. Those teachings are primarily directed toward identifying mutant genes, meaning that their primary interest is on recovering and characterizing sequences contained in the population of cells and colonies that had undergone DNA error correction of the input loop vector constructs. They presented no motivation or means to selectively recover insert sequences only from the subpopulation of plasmids containing the dysfunctional marker gene from cells containing mixed plasmids that would be less likely to contain mutations. To them, such plasmids bearing the dysfunctional version of the selectable marker gene would represent precisely the pool from which they would selectively avoid recovering the insert genes.

Together, the examples shown below demonstrate that intensification of the GFP signal in the test vector systems described here correlates with co-replication of separate plasmids derived from each strand of the input test vector. The intensified GFP signal in such colonies can serve as an indirect indicator that the test vector in those colonies had not been acted upon by the DNA repair system, and thus, that the inserted sequences were less likely to contain mutations that may have otherwise triggered the mismatch repair system and co-repair at the loop sequence. This unexpected result is particularly surprising since the GFP gene and its controlling sequences were identical between the two plasmids and would thus not have been expected to give rise to different levels of fluorescent signal in cells supporting the co-replicating plasmids when compared to those containing only the plasmid with the wild-type beta lactamase gene. This surprising observation pointed to the practical utility of the observed but unexplained intensification of colony fluorescence as a basis for identifying colonies more likely to contain error-free sequences in the DNA inserted into the test vector.

The test vector was constructed in hemimethylated form in order to provide the directional signal to the error correction system to eliminate the five nucleotide loop in the beta lactamase gene during co-repair events. If the test vector system were to perform perfectly in this regard, the E. coli methyl-directed repair system would have been expected to cause inactivation of the beta lactamase gene with consequent failure to form a colony each time a given test vector construct was co-repaired at the loop site. This would allow a colony to form only when the DNA repair system had not engaged and co-repaired the input test vector construct, thus producing a bright colony containing the two co-replicating plasmids with a higher likelihood of containing an error-free gene. As a potential explanation for the occurrence of regular-fluorescence colonies containing only a single plasmid in the above examples, the prospect of passive loss of the deletion-containing plasmid from cells that had initially supported co-replication of both plasmids was determined to be unlikely, as the perfect GFP control (Q5-GFP in Examples 1-4) and perfect M1 gene fragment (Q5-M1 in Examples 5 and 6) showed predominantly bright colonies when cloned into the test vector, indicating that persistent co-replication of the plasmids within such colonies is a stable condition when error free sequences are inserted into the test vector.

Despite the known preference described in the literature for proteins involved in the methyl-directed repair process to act upon the unmethylated strand of a hemimethylated duplex, the presence of a significant number of fluorescent colonies with only normal levels of fluorescence on plates that had originated from transformation with test vectors containing error-containing synthetic GFP genes (as described in Examples 1-4) or the mutated M1 gene (Examples 5 and 6) suggested that strand discrimination during DNA repair and co-repair may not have occurred in each instance with the expected directionality with regard to the methylated strand.

In considering how colonies with normal levels of fluorescence and only a single replicating plasmid had arisen in the test vector-mutant gene combinations, it may be more likely that fully-unmethylated GATC sequences known to be present within the inserted GFP gene (the synthetic GFP gene in Examples 1-4, and Q5-GFP gene) or within the unmethylated Taq3-M1 gene, which contains a single GATC sequence (Examples 5-7) had introduced strand-ambiguous signals for initiation of repair. Whichever the cause in this instance, imperfect strand-directionality of repair/co-repair appeared to create a background population of colonies derived from molecules that had undergone error correction and co-repair of the wrong (methylated) strand (herein called “wrong-strand repair”), leading to retention of only the wild-type beta lactamase gene on the resulting co-repaired plasmid. Such colonies would exhibit only normal levels of fluorescence, so the fortuitous and unexpected observation that colonies supporting both plasmids (wild-type beta lactamase and the five basepair deletion of beta lactamase) exhibiting brighter fluorescence provided a novel way to discriminate against this category of spurious wrong-strand repair events that had given rise to regular fluorescence colonies, and to allow for more efficient identification of error free molecules that had arisen without prior co-repair on either strand.

It is also possible that partial plasmid repair events not extending around the entire plasmid sequence could have occurred without accomplishing co-repair on either strand of the test vector loop. In the potential case of such “partial repair” events, if strand excision was initiated in either direction on the plasmid relative to the mutant insert but did not extend all the way to the portion of the plasmid containing the loop, then the sequence difference at the site of the loop in the two strands might be expected to persist. Such persistence could give rise to two co-replicating plasmids after DNA repair, which may in turn be expected to lead to intensification of the GFP marker signal, causing the generation of false positives. To address such inefficiencies, several potential strategies or potential combinations thereof could be used:
Cluster additional hemimethylated GATC sequences near the loop sequences to maximize the initiation of repair events near the loop sequences.
Remove unnecessary GATC sequences elsewhere in the plasmid construct that are not near the loop sequences to increase the likelihood of repair events initiating closer to the loop sequences.
Ensure that GATCs occurring near the loop sequences are hemi-methylated on the desired strand while fully-methylating GATC sequences occurring elsewhere in the plasmid construct to increase the chances for repair events initiating near the loop sequences.
Modify the host cell or conditions in which it is maintained in such a way as to increase the average length of excision and resynthesis tracts created by the DNA repair system to maximize the likelihood of co-repair of the loop sequence.
Minimize the DNA sequence distance between the loop sequences and the target gene containing potential DNA mismatches in order to maximize the likelihood of co-repair of the loop sequence. Such minimization might be accomplished by reducing the size of the plasmid vector. Such minimization might also be accomplished by the use of a nanoplasmid system as used by Suschak et al. (PMID: 32296729).
Modify the plasmid, host cell, or culture conditions to delay or otherwise control the timing (by controlled induction for instance) of initiation of plasmid replication after the transformation step in order to provide additional time if needed for the DNA error correction system to act on error-containing molecules.

Detection of a mismatch by MutS leads to the formation of a MutS/MutL complex on the DNA that travels along the DNA, possibly by diffusion along the double helix, away from the mismatch and toward eventual encounter with one or more GATC sites. It is possible that repair events initiating at GATC sites not near enough to the loop in the beta lactamase gene to include the loop into the repair tract could give rise to repair events that do not effect co-repair on either strand at the site of the loop. Potential repair events of this kind that would leave the loop structure intact could result in a repaired test vector that could still give rise to co-replication of two plasmids, with and without the functional beta lactamase. Such events would contribute to the level of undesirable background within the desired population of test vector molecules not undergoing any repair at all.

To avoid background of this type, it may be useful to limit the diffusion of the MutS/MutL complexes along the DNA so that they move primarily in one direction from the insertion site of the synthetic DNA fragment, and preferentially toward the loop in the beta lactamase gene, thereby increasing the likelihood that the loop would be included in any repair events occurring on the molecule. Potential ways to limit the diffusion of the MutS/MutL complexes in this way can include situating a lac operator to flank the synthetic fragment insertion site opposite the side that has the shortest path (and preferably with the fewest intervening GATC sequences) to the loop in the beta lactamase gene. Binding of a lac repressor protein to the lac operator in the plasmid can sterically impede the diffusion of the MutS/MutL complexes away from the desired direction of the loop. Hanne, et al., (PMID: 30072380) provide for the experimental use of the lac operator/repressor system in the presence of MutS/MutL complexes to study their interactions on DNA. Other ways to sterically impede the directional diffusion of MutS/MutL can include other DNA binding sequences or incorporation of biotin-labeled nucleotides that can be bound with streptavidin to create the steric block.

Such steric signals providing directionality to the action of the methyl-directed DNA repair system can be included so that control of their binding can be manipulated by environmental conditions including exposure to chemicals such as IPTG, in the case of the lac repressor, that would alter the binding between the DNA binding protein and the DNA to regulate its degree of steric influence on the MutS/MutL system. Moreover, flanking the insertion site with different binding sites for different binding proteins responsive to different environmental stimuli or chemicals can provide a degree of control over the directional and functional outcome of subsequent repair events in a way that can integrate external information into subsequent co-repair event(s) occurring as a result of engagement of the mismatch repair system on the molecule. One mode of error reduction in a population of DNAs involves the use of mismatch endonucleases to cleave DNA at mismatches created by non-consensus sequences. In such ablative error reduction processes, called DirectASE™ herein, DNA errors can be reduced by at least several fold by incubating a mixture of homoduplex DNAs and mutation-containing heteroduplex DNAs in the presence of a mismatch endonuclease. If performed on a circular DNA input, heteroduplex DNAs are more likely to become linearized than homoduplexes, which allows selective expansion and recovery of still-circular DNAs by transformation and replication in E. coli or another suitable host. If performed on a linear DNA input, fragmentation of the DNA reduces the efficiency with which the resulting fragments can be directly recovered by various means, or with which it can be incorporated into a cloning vector and subsequently cloned. In the case of linear DNAs, exonuclease activity of the mismatch endonuclease may lead to loss of terminal sequences used for amplification or cloning. This undesired trimming of termini can be reduced by incorporation of nuclease-resistant (locked-nucleotide) nucleotides into the termini, or by including ‘sacrificial’ sequences of about 10-50 nucleotides at the termini of those linear DNAs. Mismatch endonucleases also nick non-mismatched duplex DNA sequences to some extent. This undesirable background activity can limit the efficiency of such methods, so to counter it, co-incubation of the DNA and mismatch endonuclease mixture with a high-fidelity DNA ligase such as E. coli DNA ligase (along with sufficient quantities of its cofactor) can efficiently re-seal nicks made in duplex regions of the DNAs, but not nicks made at mismatch sites.

The DNA loop sequences present in the looped-heteroduplex test vector systems described here would be expected to be highly susceptible to cleavage by the mismatch endonuclease used for DirectASET processes, making it seemingly impractical to use a loop-containing test vector construct in a DirectASE™ reaction. However, preventing cleavage of the loop sequences by the mismatch endonuclease in a DirectASE™ reaction could allow the two techniques to be used simultaneously. Reducing or preventing cleavage of test vector loop residues in the presence of mismatch endonuclease may be accomplished by a number of means, including incorporating nuclease-resistant nucleotide linkages at or near the loop(s) to prevent cleavage in the DirectASE™ reaction, or by blocking mismatch endonuclease access to the loop itself. Blocking access could be accomplished by incorporating a DNA binding protein recognition sequence(s) near the loop so that binding of the DNA binding protein near the loop region would compete with the mismatch endonuclease and block its access to the loop. As with the introduction of steric blockages as described above to provide directional signals to the DNA repair system, blocking access to the loop could be accomplished by various means. Such means include, binding of a cleavage-deficient mismatch endonuclease to its DNA recognition site as described by Pluciennik and Modrich in PubMed PMID: 17620611, incorporating a LacO site and binding it with the Lacl repressor as described in PubMed PMID: 30072380, and by targeting cleavage-deficient Cas9 protein (dCAS9) to the site as described by Mardenborough, et al. in PubMed PMID: 31598722. An additional method for blocking mismatch endonuclease access to the loop could include incorporating biotinylated (or desthiobiotynylated) nucleotide residues through various means, including nick-labeling, and binding the modified DNA with streptavidin. If desthiobiotin is used, it may be possible to dissociate the streptavidin from the DNA after the DirectASET reaction by incubating the complexes in the presence of biotin, either in vitro or in vivo.

By utilizing the unexpected insights presented here that error-free molecules can be identified more efficiently by selecting the brighter category of fluorescent colonies, it may be possible that the test vector constructs need not necessarily be hemimethylated to achieve similar discriminatory effect to identify error-free sequences. Test vector constructs could potentially be either fully methylated, partially methylated on either strand, or not methylated at all, but with potentially different outcomes in terms of the degree of error reduction obtained. The strand of the test vector described here that encodes a nonfunctional beta lactamase contains a five base pair deletion that abolishes its function. Deletions larger than five base pairs were also evaluated in similar test vector constructs with similar results, though test vector constructs containing a very large deletion of over 200 basepairs appeared to undergo spontaneous resolution from heteroduplexes to homoduplexes upon storage. The test vector system could work with other selectable markers and has also been shown herein to function with high efficiency to identify error-free genes when combined with a screenable marker gene in the same test vector that contains a wild-type strand and a deletion in the other strand that eliminates the function of the resulting gene product.

The following nonlimiting examples are provided to illustrate the present invention.

### Example 1

A sample of linear hemi-methylated double stranded plasmid vector DNA containing a five nucleotide loop in the beta lactamase gene was prepared and named “test” vector. This double-stranded loop-containing vector DNA was formed using heat denaturation and annealing to pair strands of an unmethylated plasmid DNA sample from pMM12, which contained a full-length beta lactamase gene, with complementary strands of methylated DNA (dam methylated at GATC sequences) from pMM13, that contained a five nucleotide deletion in the beta lactamase gene that rendered the gene nonfunctional. The unmethylated pMM12 plasmid was produced using a DNA methylase-deficient E. coli strain, and fully methylated pMM13 was prepared in DH5alpha cells. Each plasmid was linearized by digestion with restriction enzyme AsiSI. The two linearized DNAs were mixed in equal proportion in Cut Smart Buffer (New England Biolabs), heat denatured by incubation at 98° C. for 4 minutes, followed by immediately shifting the temperature to 0° C. for 5 minutes, and annealed by incubation at 37° C. for 5 minutes before placing the resulting double stranded DNA mixture on ice. This double-stranded DNA preparation contained hemimethylated double-stranded heteroduplex DNAs with a five nucleotide loop in the beta lactamase (ampicillin resistance) gene (test vector) as well as re-annealed DNAs corresponding to the original input DNAs (pMM12 and pMM13). The mixture was treated with MboI and DpnI to degrade the fully nonmethylated and fully methylated homoduplex DNAs into smaller fragments, leaving the hemimethylated test vector DNA intact. The intact test vector was subsequently purified away from the smaller fragments by size fractionation. As a control, a fully methylated pMM12 plasmid sample (pMM12-M), containing the wild-type beta lactamase gene was also prepared as a no-loop comparison. With the exceptions of being fully methylated and having no loop, this control vector was identical to the test vector.

The resulting double stranded, hemimethylated, heteroduplex vector DNA was similar in concept to that produced by Yehezkel et. al (PMID: 23155373) and Cox (PMID: 8808468), in that insertion of error-containing sequences into the plasmid vector would be expected to be engaged by the E. coli DNA repair system to initiate repair upon the error-containing plasmid. Loops of about five nucleotides and larger are known not to be recognized by this repair system, so if no mutations that formed DNA mismatches were introduced by the inserted sequence, the in vivo methyl-directed mismatch repair system would not be engaged, allowing both strands of the test vector to be replicated. This would be expected to allow the beta lactamase activity encoded by the one plasmid to facilitate growth of the cell on selective medium containing ampicillin or carbenicillin and provide the opportunity for the deletion-containing plasmid to co-replicate in the same cell. In the case of an insert sequence containing one or more DNA mismatches or small insertion/deletion errors, the DNA repair system would be engaged upon its introduction into the E. coli host. Cleavage and excision of a sufficient tract of sequences from the non-methylated strand would be expected to eliminate the loop formed in the beta lactamase gene via ‘co-repair’, forming a deletion on both strands of the beta lactamase gene, making it unable to support growth of the host cell on ampicillin or carbenicillin media. This would ultimately be expected to lead to elimination of the co-repaired plasmid from the population of transformed cells.

A synthetic gene product encoding the GFP gene was prepared using PCR overlap assembly from a pool of overlapping 60-mer oligonucleotides that had been purchased from an oligonucleotide synthesis company in about 2009, and that was known to give rise to a synthetic GFP gene containing a significant number of mutations, including insertions and deletions of various sizes, as well as base substitutions. An aliquot of the resulting synthetic GFP PCR product was heat denatured and complementary strands were allowed to anneal, forming a mixture containing double-stranded heteroduplexes in which at least one of the annealed strands contained an error, and homoduplexes in which primarily error-free sequences had annealed to one-another. The resulting mixture was cloned into the test vector as well as the pMM12-M control vector via Golden Gate Assembly (GGA) using BsaI (NEBE1601). Both vectors contained a promoter situated at the junction with the inserted fragment to direct the expression of the GFP gene in E. coli. As a control, an error-free GFP fragment was prepared from a clonal isolate of the GFP gene using PCR with the high-fidelity Q5 polymerase. This “Q5-GFP” control fragment was cloned into both vectors in parallel with the synthetic GFP heteroduplex/homoduplex mixture. The resulting ligation products of the four GGA reactions were transformed separately into E. coli cells (NEBC29871) according to manufacturer's instructions, with the exception that post heat shock recovery was performed for 1 hour at 25° C., and then plated onto LB-amp agar plates. After overnight incubation at 37° C., colonies were scored for the presence or absence of green fluorescence under longwave UV light, revealing fluorescent and non-fluorescent colonies.

According to Yehezkel and Cox, the relative proportion of fluorescent and non-fluorescent colonies would be expected to vary as a function of the error rate of the synthetic GFP gene product; a higher percentage of fluorescent colonies correlates with lower overall error rates of the GFP genes represented in the population of colonies. Consistent with that correlation, cloning of the error-containing population of synthetic GFP genes into the test vector preparation produced a higher percentage of green fluorescent colonies than those cloned into the unmodified pMM12-M control vector (Table 1). In both vectors, the control “perfect” GFP homoduplex gave rise to predominantly, if not exclusively, green colonies.

TABLE 1
Percent green fluorescent and non-fluorescent colonies
resulting from cloning of either synthetic or “perfect” GFP
genes into either the test vector or pMM12-M control vector

| | | Insert | |
|---|---|---|---|
| | | Synthetic GFP gene | |
| | | hetero/homo-duplex | “perfect” GFP control |
| | | insert | insert |
| Vector | Test | 70% fluor, 30% non-fluor | >99% fluor, <1% non- |
| | vector | (bright colonies and | fluor |
| | | normal colonies) | (nearly all bright colonies) |
| | pMM12-M | 45% Green, 55% non- | >99% Green, <1% non- |
| | control | fluor | fluor |
| | vector | (no bright colonies) | (no bright colonies) |

Numerous experiments were performed utilizing additional methods for constructing hemi-methylated loop-containing test vectors, with similar outcomes that showed increased percentages of fluorescent colonies and corresponding reductions of errors in the resulting cloned populations of GFP genes. When measured by DNA sequencing of cloned GFP genes derived from randomly selected colonies from such experiments, the presence of DNA errors was typically observed to be reduced by three to five-fold when compared to the same GFP gene sample cloned into the pMM12-M control vector.

Interestingly, and as indicated in parentheses in Table 1, it was noted that when observing the plates from the above experiment under longwave UV illumination, a significant fraction of colonies resulting from the synthetic GFP hetero/homoduplexes cloned into the test vector emitted discernably more intense (brighter) fluorescence than those on the plate in which the same fragment had been cloned into the pMM12-M control vector. The same was true for the “perfect” GFP fragment, except that nearly all those colonies were brighter when cloned into the test vector than when cloned into the pMM12-M control vector. However, all of the fluorescent colonies resulting from insertion of either GFP fragment into the pMM12-M control exhibited normal levels of fluorescence, with none showing the brighter level of fluorescence that was observed to be associated with the test vector colonies. No overall differences were expected to be present in the respective GFP genes or their control sequences in DNAs derived from the bright versus regular colonies resulting from the test vector versus the pMM12-M control, so this unexpected result was pursued to understand the observed differences in colony fluorescence intensity.

### Example 2

To explore the unexpected presence of more intensely fluorescent colonies in addition those showing the expected normal levels of fluorescence in the test vector plates, a brightly fluorescent colony from the test vector/synthetic GFP plate and a colony with regular fluorescence from the pMM12-M/synthetic GFP control plate was picked, grown in liquid culture for plasmid extraction, and DNA sequencing (Sanger) was performed to obtain the entire plasmid sequence for each. The GFP gene sequence showed no errors in either plasmid. In the plasmid backbone sequences, no sequence differences were observed between them, except that the sequence traces from the bright colony plasmid showed a strong signal for the five base pair beta lactamase deletion overlayed upon a weaker signal corresponding to the wild-type beta lactamase gene. These overlapping sequence chromatograms corresponded to the input plasmid DNA strands from pMM-13 and pMM-12, respectively, that were used to produce the test vector, and correspondingly, to each of the opposite strands (representing the five base pair deletion and the wild-type beta lactamase genes) present in the test vector upon introduction into E coli cells. This sequencing result indicated that each of the individual strands in the test vector had given rise to plasmid DNAs in the mixed plasmid preparation obtained from the bright colony. Streak-isolation was performed onto fresh media from individual bright colonies and the resulting colonies were found to contain the same mixture of the two parent plasmids, indicating that the two plasmids were co-replicating in the same cells rather than replicating exclusively in separate cells within a given colony. Two different mixed plasmid preps derived from bright colonies, were then transformed into E. coli and plated on nonselective agar medium. Fluorescent colonies arising on those plates were picked and patch-inoculated onto both nonselective and selective media, resulting in approximately 75% and 90% of the patched cultures growing on nonselective LB agar but not on LB agar containing carbenicillin. This result indicated that the majority of plasmid material (approximately 75% and >90%) in the plasmid preps isolated from individual bright colonies corresponded to the beta lactamase deletion plasmid. It was expected that if either plasmid in such mixed-plasmid cells and cultures might have had any growth advantage over the other, it might have more likely been the plasmid with the functional beta lactamase gene, so these results to the contrary were surprising. Overall, these results support the conclusion that the two plasmids co-replicate in cells, and that the deletion plasmid appears to be disproportionately represented in those cells. This observed higher relative abundance of the beta lactamase deletion plasmids in cells supporting their co-replication may have contributed to their evident ability to persist in those cells.

### Example 3

To determine the actual error frequency of the different categories of colonies (bright-fluorescence, normal fluorescence, no fluorescence) resulting from the previous examples, colonies from each category were picked and their GFP genes were PCR amplified and sequenced. In all, 35 sequences were obtained from brightly fluorescent colonies, 33 sequences were obtained from normal fluorescence colonies, and 24 sequences were obtained from nonfluorescent colonies. For the brightly fluorescent colonies, 34 of 35 sequences were determined to be perfect sequences with no mutations in the GFP gene, and one sequence contained a single mutation, for an error rate of approximately one mutation per 25,000 bases. Of the colonies showing normal fluorescence, 24 of 33 were perfect and nine had mutations for a total of approximately one mutation per 2,400 bases. Of the non-fluorescent colonies, all 24 were mutated with an error rate of approximately one in 450 bases. It was expected that all non-fluorescent colonies in this experiment would be mutated since the synthetic gene in this case also served as the readout for the fluorescence-based picking step. In any event, the much lower mutational load in the GFP genes obtained from the brightly fluorescent colonies relative to the normal fluorescence category was notable and indicated that sequences with fewer errors could be identified from the population of colonies with bright fluorescence.

### Example 4

Competent E. coli cells were transformed and plated as described in the previous examples but incubated over the weekend at room temperature (˜20 to 25° C.). After this incubation, the colonies were illuminated under longwave UV light and fluorescence was observed, but unlike the earlier experiments in which the colony plates had been incubated at 37° C., brightly fluorescent colonies could not be discerned. The plates were then incubated at 37° C. for an additional eight hours and reinspected under longwave UV light. As expected, the colonies had grown somewhat during 37° C. incubation. It was also noted that a subset of colonies from the test vector/synthetic GFP gene transformation exhibited increased fluorescent brightness at their periphery, suggesting that incubation temperature had influenced the development of higher fluorescence in those colonies.

### Example 5

In the previous examples, the GFP sequence that was inserted into the test vector served as both the trigger for DNA mismatch repair as well as the screenable marker to discriminate between colonies supporting co-replication and those supporting only plasmid with the functional beta lactamase gene. In order to separate the fluorescence-based reporter activity of the GFP gene from the sequences triggering engagement of the DNA error correction system, another set of experiments was conducted in which two DNA fragments were inserted into the test vector. One of the fragments encoded a non-mutated GFP gene, called Q5-GFP, that had been generated by PCR from a clonal GFP isolate using Q5 high-fidelity DNA polymerase and low PCR cycle numbers to minimize the presence of mutations in the resulting fragment. This gene was included in each of the subsequent GGA reactions as a fluorescent readout ‘sensor’ screenable marker gene to determine whether higher fluorescence intensities would also be observable in this context to allow for discrimination of brighter colonies containing co-replicating plasmids from regular-fluorescence colonies expected to contain only the single plasmid encoding wild-type beta lactamase. The second fragment served as the mutation-containing “trigger” to activate in-vivo DNA error repair when mismatches were present. Called “Taq3-M1”, this fragment was a portion of an influenza virus M1 gene that had been amplified through numerous rounds of PCR using Taq DNA polymerase to introduce mutations, primarily single nucleotide base changes, into the amplified fragment. After heat denaturation and reannealing of the Taq3-M1 gene to allow heteroduplex formation from mutation-containing strands, three-piece GGA assembly reactions were performed to insert both the re-annealed Taq3-M1 trigger gene and the Q5-GFP sensor gene into the test vector as well as the pMM12-M control vector. A non-mutant control was also performed in which a non-mutated version of the M1 gene (“Q5-M1”) was prepared using Q5 polymerase and inserted into the test vector. On its 5-prime end, the Q5-GFP gene was positioned identically relative to the promoter driving its expression as in the previous examples. The mutated Taq3-M1 gene (or its non-mutated control) was designed to be inserted immediately downstream of the GFP at the junction where the GFP gene had been joined to the test vector in the earlier examples. Thus, the M1 trigger was sandwiched between the loop-containing beta-lactamase gene and the GFP sensor gene.

The GGA reactions were transformed into competent DH5alpha E. coli, recovered at 25° C., and plated in duplicate on LB-carbenicillin agar plates. One set of plates was placed at 37° C., and the duplicate set was placed at 40° C. After overnight incubation, the plates were examined under longwave UV illumination, and it was noted that fluorescence intensity differences among colonies on the plates incubated at 40° C. were clearly discernable, whereas the colonies on the plates incubated at 37° C. did not exhibit such differences in fluorescence. Colonies were picked according to fluorescence intensity (brighter versus regular fluorescence levels) from the 40° C. plates, the region spanning the M1 gene and the five nucleotide loop-forming deletion of the beta lactamase gene was PCR amplified from each, and the resulting products were sequenced to count the errors present in the M1 gene and to assess whether the colonies contained both plasmids (the wild-type as well the five basepair-deleted beta lactamase plasmids) or only the plasmid containing the wild-type beta lactamase. The results were as follows: All the colonies emitted GFP fluorescence, which was expected since the Q5-GFP sensor fragment used in this example was a perfect non-mutant homoduplex in both GGA assembly reactions. The sequence data agreed with the earlier results that all the brighter colonies contained both plasmids (one encoding the five basepair deletion in the beta lactamase gene, and the other containing the wild-type non-deleted version of the gene), while the colonies with regular fluorescence contained only the plasmid encoding the wild-type beta lactamase gene. The Taq3-M1 fragment inserted into the pMM12-M control vector along with Q5-GFP showed an error frequency of 1 in 533 bases. The brighter-fluorescence colonies picked from the plate containing the Taq3-M1 fragment inserted into the test vector along with Q5-GFP showed an error frequency of approximately 1 in 3,200 bases. The normal-fluorescence colonies picked from the same plate showed an error frequency of approximately 1 in 400 bases. Thus, an eight-fold decrease in DNA errors was obtained by employing the insights gained above to select the brightly fluorescing colonies and exclude the colonies showing normal fluorescence. The control reaction in which the non-mutated Q5-M1 was inserted into the test vector along with the Q5-GFP resulted in nearly all colonies emitting bright fluorescence. These results agreed with the results described in the previous examples. This experiment demonstrated that the unmutated (perfect) GFP gene could function as a separate ‘sensor’ component when added to the test vector to enable the identification of colonies supporting the replication of both plasmids, and that such colonies contained far fewer mutations in the M1 gene than the category of regular-fluorescence colonies that had been determined in the above examples as likely to represent wrong-strand repair events.

### Example 6

The GGA assembly reaction containing the test vector, the mutated Taq3-M1 gene “trigger”, and the unmutated Q5-GFP “sensor” described in the previous example was re-transformed into E. coli, plated in triplicate on LB agar plates containing carbenicillin, and incubated at either 37° C., 40° C., or 42° C. After overnight incubation, the plates were examined under longwave UV illumination. Consistent with the results of the previous example, fluorescence intensity differences among the colonies were much more pronounced on the plates incubated at 40° C. and 42° C. than at 37° C., with the plates incubated at the higher temperatures showing distinctly brighter-fluorescence category of colonies than when incubated at 37° C. Colonies were picked according to fluorescence intensity from the 40° C. plate, and the region spanning the M1 gene was PCR amplified from each and sequenced to count the errors present in the M1 gene. Colonies from 42° C. plate were not picked, as one side of the plate had dried during incubation and relatively few colonies were deemed suitable for picking. The results were as follows: As observed in the previous example, all the colonies emitted GFP fluorescence. The brighter-fluorescence colonies representing the Taq3-M1 fragment inserted into the test vector along with Q5-GFP showed an error frequency of 1 in 5,776 bases, with 34 of 38 sequences having no mutations. The category of normal-fluorescence colonies picked from the same plate showed an error frequency of approximately 1 in 800 bases, with only 18 of 38 having no mutations. Thus, an eight-fold decrease in average DNA error frequency was obtained by selecting the brightly fluorescing colonies instead of the colonies showing normal fluorescence, confirming the beneficial effect of using GFP fluorescence intensification to guide the selection of a population of higher quality, lower-error genes.

### Example 7

In the previous examples, a GFP gene was used as a “sensor”. In this example, a red fluorescent protein gene that also contained a 5 base deletion loop similar to that in the beta lactase gene, is used as a “sensor”. However, instead of bright green colonies indicating co-replicating plasmids, in this case dim red colonies indicate co-replicating plasmids.

A sample of two hemimethylated linear double stranded plasmid vector DNA fragments, named “LS-amp-ori” and “XFP_A”, was prepared. “LS-amp-ori” contained a 5 base loop in the beta lactase gene. “LS-amp-ori” was prepared by annealing unmethylated single-stranded DNA containing a wild-type beta lactamase gene with methylated single-stranded DNA containing a 5 base deletion in the beta lactase gene. “XFP_A” contained a 5 base loop in the red fluorescent protein (RFP) gene. “XFP_A” was prepared by annealing unmethylated single-stranded DNA containing a functional RFP gene with methylated single-stranded DNA containing a 5 base deletion in the RFP gene. Unmethylated double stranded DNAs were prepared by PCR amplification with the appropriately modified primers from plasmid DNAs containing the respective vector fragments of interest. Methylated double stranded DNA was prepared by exposing unmethylated double-stranded DNA to dam Methyltransferase using a commercial kit (New England Biolabs). Single stranded DNA was prepared by exposing double stranded DNA containing a series of nuclease-resistant “locked” nucleotide linkages between several of the 5′ terminal residues of one strand, and a 5′-terminal phosphate group and no locked nucleotides on the complementary strand, to lambda exonuclease, which selectively degrades the 5′-phosphorylated strand without 5′ locked nucleotides. The locked nucleotides and 5′-phosphate were incorporated by inclusion of the appropriate primer pairs used for amplification of the respective LS-amp-ori and XFP_A fragments. As a control, fully methylated plasmid containing a wild-type beta lactase gene, but no red fluorescent protein gene, was prepared as a no-loop comparison. “LS-amp-ori” and “XFP_A” also contained a terminal restriction site to generate cohesive overhangs to allow the two fragments to be joined together by GGA such that once joined, one DNA strand would be fully methylated and the other strand would be fully unmethylated.

A third fragment called “Taq4-M1”, was used as the insert fragment after a melt-anneal cycle to generate a mixture of dsDNAs containing either perfectly base-paired sequences or containing random DNA mismatches to “trigger” methyl-dependent DNA mismatch repair upon transformation into E. coli. “Taq4-M1” is identical to “Taq3-M1” in EXAMPLE 5 except that “Taq4-M1” was amplified through additional rounds of Taq PCR and contains compatible cloning ends with “LS-amp-ori” and “XFP_A”.

GGA reactions were prepared to join the “LS-amp-ori” and “XFP_A” fragments with the “Taq4-M1” “trigger” into a circular construct. The control vector was assembled in parallel using GGA with the “Taq4-M1” “trigger”. The GGA reactions were transformed into competent DH5alpha E. coli, recovered at 25° C., plated on LB-carbenicillin agar plates. The control vector transformation was incubated at 37° C. for 2 days and the test vector transformation was incubated at room temperature for 2 days followed by 37° C. for about 7 hours.

Colonies on the plates were scored for color development as “red”, “dim red”, or “white”. A DNA fragment containing the M1 “trigger” gene was then PCR amplified from colonies and the resulting DNA sequenced by the Sanger method to measure the error rate in the M1 gene.

The results were as follows: The “Taq4-M1” cloned into the fully-methylated control vector contained an error frequency of 1 in 533 bases in the M1 gene with all colonies white due to the lack of a red fluorescent protein gene in that construct. The “Taq4-M1” cloned into the “LS-amp-ori” and “XFP-A” construct contained an overall error frequency of 1 in 2,154 bases in sequences obtained from randomly-selected colonies. Of these, the dim red colonies contained an overall error frequency of 1 in 15,200 bases. Thus, a 4-fold reduction in the error rate is obtained by selecting on antibiotic selection alone and a 29-fold improvement is obtained by further selecting for dim red color. As in previous examples with bright GFP, “dim red” colonies indicate co-replicating plasmids that arise due to the MisMatch Repair machinery not being engaged for the plasmid. Red fluorescent protein gene with a 5 base deletion results in a white colony, while red fluorescent protein with a fully wild-type gene results in a red colony. Red colonies could arise due to wrong strand repair or loss of the co-replicating plasmid in non-repaired plasmid, while white colonies could arise due to partial repair of the plasmid. Both categories were considered to represent undesirable background colonies. Therefore, in selecting for dim red colonies, most of the colonies should not have activated repair and the “trigger” is more likely to be error-free. Indeed, the red background colonies had an error frequency of 1 in 840 bases and the white colonies had an error frequency of 1 in 2,200 bases. Since the 5 base frameshift-causing deletions are on the same strand for both red fluorescent protein gene and the beta lactamase gene, colonies having only a single plasmid (by definition, those with functional beta lactamase) would also be expected to have a strong red color. In colonies containing a mixture of plasmids derived from each of the test vector input strands, the higher copy number of the plasmid containing a 5 base RFP deletion had likely caused less intense red color development than could be easily discerned if they were mixed evenly. Therefore, assuming that the unexpected copy number advantage observed in Examples 5-6 for the plasmid with the dysfunctional beta lactamase gene also extends to the highly similar construct used in this experiment, skewing of the colony color intensity toward the dysfunctional RFP and away from the functional RFP made it easier to discern the colonies in which the two plasmids are co-replicating from colonies in which only one or the other plasmid is present.

### Example 8

Selective Amplification of Error-reduced Genes from Cells, Either Clonally or in Bulk

The test vector constructs of Examples 1-6 are transformed into E. coli and grown on plates. Colonies are picked and PCR amplification reactions are performed from each. One of the PCR primers selectively anneals to and primes DNA synthesis on the strand containing the five basepair deletion in the beta lactamase gene but is not capable of priming synthesis on the strand containing the wild-type beta lactamase gene. Thus, only colonies containing the deletion vector can successfully give rise to PCR products. Consistent with the examples above, the deletion-containing plasmid is present only in cells that also contain the plasmid encoding the wild-type beta lactamase. Furthermore, as shown in the above examples, colonies supporting a mixture of both plasmids are less likely to have undergone DNA repair and co-repair and, further consistent with the above examples, are more likely to contain error-free genes. Essentially, any loop feature that permits the selective recovery of the dependent plasmid by whatever means can work.

### Example 9

An aliquot of the synthetic GFP PCR product made from the pool of 60-mer oligonucleotides as described in Example 1 was used as template for a PCR amplification reaction to introduce BsmBI sites immediately internal to its terminal BsaI sites. After insertion of this fragment into the loop-containing heteroduplex test vector preparation (called pLogic-92121 in this and subsequent examples), using BsaI for GGA, the GFP ORF would still be flanked by the BsmBI cleavage sites useful for subsequent GGA-based DNA joining without the need for an additional PCR step to add flanking cloning sites back to the ORF. This PCR was performed using the primers 2S-GFP-F (ACTTGACAGTCTGGTCTCCGATGACGTCTCAGATGAGCAAAGGAGAAGAAC TTTTCAC) and 2S-GFP-R (ATGCAGCATTGGTCTCTCTTATCGTCTCACTTATTTGTAGAGCTCATCCATG CC) with Q5 DNA polymerase (NEB) with the synthetic GFP gene template according to the manufacturer's instructions.

Following this PCR reaction, the sample was subjected to the same melt/anneal procedure as described above in Example 1 and then run on a 1% agarose gel in TAE buffer. The band representing the approximately 770 bp synthetic gene product, called “2S-GFP”, was excised and isolated using a freeze-squeeze gel extraction method. This gel-purified fragment was then introduced into pLogic-92121, as well as into pMM12 as negative control, using an NEB GGA kit (BsaI, according to the manufacturer's instructions). One microliter of each GGA reaction was then added to 20 microliters of ice-cold NEB 5-alpha High Efficiency competent E. coli cells (NEB) in 15 ml disposable polypropylene tubes, incubated on ice for 30 minutes, heat shocked at 42° C. for 30 seconds and transferred back to ice for another 5 minutes. Each tube then received 379 microliters of SOC medium and was incubated at 25° C. for 1 hr with occasional agitation.

After this step, between 1 and 20 microliter aliquots of the 400 ul post-recovery cell culture sample were plated on LB-agar containing 100 ug/ml carbenicillin. After incubation at 36° C. for 3 hours, the plates were transferred to 40 C and incubated overnight or longer to allow for colony growth and sufficient accumulation of the GFP protein to allow visualization of fluorescent colonies under UV illumination. The numbers of green fluorescent colonies were determined and expressed as a percentage of the total number of colonies arising from each of the transformations (Table 2).

The baseline frequency of functional GFP genes (individual clones giving rise to fluorescent colonies) in the starting gel-purified 2S-GFP fragment material was determined to be approximately 45% by counting colonies arising after insertion of the 2S-GFP fragment into the pMM12 negative control vector (Indicated as #1 in Table 2). The percentage of green colonies arising after insertion of the same 2S-GFP fragment into the pLogic-92129 vector was determined to be 73%, as shown by box #2 in Table 2. The difference in those frequencies illustrates the degree of error reduction obtained solely by insertion into pLogic-92129 followed by E. coli transformation and plating. Extrapolating from the numbers of colonies arising on these plates, each of the initial bacterial transformations was calculated to have produced at least several thousand independent cellular transformation events.

Immediately after plating the aliquots taken from the 400 ul post-recovery cell samples described above, 600 microliters of LB containing 133 micrograms of carbenicillin per ml was added to the remaining liquid culture derived from the pLogic-92121 GGA reaction. The culture was then shaken overnight at 37° C. A 50 microliter sample of the densely-grown overnight culture was taken and the cells were pelleted by centrifugation and resuspended in 500 microliters of TE pH 8.0. One microliter aliquots of this cell preparation (representing well-over 10,000 viable cells per microliter) were used as the source of template for PCR reactions to reamplify a segment of the plasmid containing the inserted GFP gene. Two such PCR reactions were performed.

One PCR reaction was primed with primers AmpLoop-Both (CAAAAAAGGGAATAAGGGCGACAC) and pBS-flank-F (GTTAGCTCACTCATTAGGCAC) to amplify sequences arising from both the pMM12- and the pMM13-derived plasmid lineages arising from the respective strands of the pLogic-92121 test vector. This PCR product was gel isolated as above and inserted into pMM12 using GGA with BsmBI. This GGA reaction was then used to transform competent E. coli cells which were then plated as described above. As expected, the colonies resulting from this GGA showed a very similar frequency of green fluorescence (76%, as shown by Table 2, #3) when compared to culture from which the PCR product used in this GGA had been derived (73%; Table 2, #2). We had earlier observed that in liquid cultures of clonally-isolated cells (e.g., cells picked from individual colonies) that harbored mixed pMM12-derived and pMM13-derived plasmid populations (each from its own corresponding strand of the pLogic-92121 input), plasmid DNA bearing the 5 nucleotide deletion and dysfunctional beta lactamase gene was more abundantly represented than the pMM12-derived plasmid. As also presented above, we had discovered that the synthetic GFP genes originating from cells harboring mixtures of pMM12- and pMM13-derived plasmids contained fewer errors on average than the GFP genes derived from cells with only the pMM-12-derived plasmid. To at least some extent then, the observed copy number effect may have contributed to the apparent small enrichment for functional GFP genes in #3 versus #2.

Using the same pLogic-92121-derived overnight liquid culture as the template source, a PCR reamplification reaction was performed with primer pair DelAmp-7 (GAATAAGGGCGACACTGTTG) and pBS-flank-F to selectively amplify the GFP gene-containing fragment from only those template DNA input molecules carrying the 5 bp deletion (non-functional beta lactamase) associated with the pMM13-derived strand of pLogic-92121. This primer pair had previously been shown to not generate PCR product when provided only with pMM12 as template (not shown). This selective ability to PCR amplify sequences only from the pMM13 lineage carrying the 5 bp deletion provided a means to discriminate in favor of a pool of GFP sequences with fewer average errors (as judged by the percentage of functional GFP genes) than the analogous non-discriminatory PCR product described in the preceding paragraph.

The product from this selective PCR reaction was gel isolated as above and inserted into pMM12 and into pLogic-92121 by GGA using BsmBI. E. coli cells were transformed with the resulting GGA reactions and aliquots were plated on LB-Agar with Carbenicillin 100 and incubated overnight as described for the plates above. Colonies arising from the GGA of the selectively-amplified product with pMM12 showed a substantially higher frequency of green fluorescence (89%; Table 2, #4) than colonies derived from the parallel nonselective PCR reaction (76%; Table 2, #3), or from the earlier culture that was used as the template source for the selective PCR (73%; Table 2, #2). These results established that DNA errors were indeed lower in the selectively amplified PCR product than in its non-selectively amplified counterpart. Further, 97% of colonies were green when this selectively amplified product was inserted into pLogic-92121 and transformed and plated (Table 2, #5), indicating that at least one additional round of cloning into pLogic-92121, with subsequent exposure to the E. coli DNA repair system and replication of resulting plasmid(s) can bring about additional reduction of errors in the target gene sequences.

DNA sequence analysis was performed from colonies corresponding to the untreated control #1 in Table 2 to obtain a baseline error rate for the uncorrected synthetic GFP fragment. Of sixteen randomly picked colonies from the no-treatment control, eight were fluorescent and eight were nonfluorescent under UV light. Of the 8 fluorescent genes, seven had no errors in the GFP gene and one had a single base change. As expected, of the 8 nonfluorescent colonies, all had errors, mostly small indels. In total, the population of 16 randomly selected genes contained a total of 17 mutations, for an error rate of roughly one in 675 bases. Note that earlier experiments based on the use of the same synthetic GFP test fragment showed a baseline error rate of about one in 450 bases, so the actual uncorrected error rate may lie somewhere between those two values.

96 green fluorescent colonies corresponding to Box #5 in Table 2 were also picked and sequenced. 93/96 contained no errors. All three mutants in this set had single base changes. The green colonies and non-fluorescent colonies from the control plates (corresponding to Table 2, #1) and the most fully-treated plate (corresponding to Table 2, #5) were picked and sequenced in separate lots, and were thus not picked randomly with regard to fluorescence. Nonetheless, these DNA sequencing data can be combined with the observed GFP colony fluorescence value of 97% green colonies to calculate an approximate 12-to-20 fold-improvement in the error rate (depending on the actual error rate of the initial uncorrected fragment) between the GFP fragment populations analyzed in #1 versus #5 of Table 2.

By optimizing various of these steps, such as by optimizing the selective PCR (SelectAmp) conditions, or by utilizing a second looped site to permit additional discrimination by the opposing primer, for instance, it is reasonable to expect that additional error reduction gains can be realized. Additional optimizations could also include changes to procedural steps described here, such as changes in time, temperature, or host organism. It is also possible that instead of exposing the heteroduplex DNAs described here to the DNA repair system and then expanding the plasmid population in vivo as described here, it is plausible that the system could be modified so that exposure with the DNA repair system and even subsequent amplification and recovery of the desired reduced-error DNA strands may be performed in vitro using a cellular extract or cocktail of defined components.

| | 2S-GFP PCR product | | | | |
|---|---|---|---|---|---|
| Insert DNA: | [symbol] | [symbol] | | | |
| 1st Cloning Vector | pMM12 (#1: 45%) | p92121 (#2: 73%) | | | |
| ↓ | Negative control | [symbol] | | [symbol] | |
| Reamplification PCR: | | Non-discriminating | | Discriminating | |
| ↓ | | primers | | primers | |
| | | [symbol] | [symbol] | [symbol] | [symbol] |
| 2nd Cloning Vector: | | pMM12 | N/D | pMM12 | p92121 |
| | | ↓ | | ↓ | ↓ |
| | | (#3: 76%) | | (#4: 89%) | (#5: 97%) |

### Example 10

In another experiment, the 2S-GFP gene was first subjected to enzymatic DNA error reduction by treatment with the ErrASE enzyme (essentially according to Kosuri et. al., PMID: 21113165), then inserted into pLogic-92121 using GGA and BsaI followed by E. coli transformation and overnight liquid culture of the entire transformation mix. A round of selective amplification was then performed using the overnight culture as template along with primers DelAmp-7 and pBS-flank-F, and the resulting PCR product was then inserted into pLogic-92121 via GGA using BsmBI, followed by E. coli transformation and overnight liquid culture of the entire transformation mix in LB-Carb, all as described above. From this second overnight liquid culture, another round of selective amplification with DelAmp-7 and pBS-flank-F was performed, and then used as template in a subsequent PCR reaction using primers GFP-GGamp-F (acttgacagtctggtctccgatgagcaaaggagaagaacttttcac) and GFP-GGamp-R (gtatgcagcattggtctctcttatttgtagagctcatccatgcc) to reintroduce BsaI cloning sites to the termini of the GFP gene-containing fragment. After gel isolation and GGA into pLogic-92121 using BsaI, cells were plated and the resulting colonies were examined under UV light to determine the percentage of functional GFP genes, as judged by the percentage of fluorescent colonies. 96% of the resulting colonies emitted green fluorescence under UV light. Eight non-fluorescent colonies were picked and sequenced. Of those eight, one gave no sequence trace, and of the other seven, none contained any piece of the GFP gene insert, with three being completely empty with small terminal vector deletions, and four containing pieces of a sacrificial DNA sequence that had carried over as a trivial artifact of pLogic-92121 heteroduplex DNA preparation process.

The final population of GFP genes resulting from this experiment had been put through a conventional enzymatic error reduction step followed by three rounds of insertion into pLogic-92121 with two intervening rounds of selective amplification. Because of the absence of any piece of GFP sequence in the set of non-fluorescent colony picks, it was concluded that within the final pool of GFP fragments, nonfunctional GFP genes were sufficiently rare that they weren't represented at all in the small number of nonfluorescent colonies analyzed here. Thus, it was concluded that when the error reduction steps described here are run iteratively, and run in series with orthogonal DNA error reduction steps, very low error rates can be achieved even from populations of DNAs with very high initial error rates (roughly 1 in 450 to 1 in 675 bases in the control experiments shown herein).

The methodologies and products presented herein can be employed in a variety of workflows. These include manual kit-based procedures conducted in individual researcher labs, industrial gene synthesis workflows based on highly automated DNA synthesis capabilities, and for increasing the quality of DNA output from benchtop DNA synthesis instruments.

These methodologies represent only specific examples and should not be taken to strictly demarcate the bounds of the claimed invention. Indeed, many potential alternatives for each component, functionality and step of the claimed invention would likely come to the mind of any person having ordinary skill in the art.

What is claimed is:

1. An in vitro method of isolating an unmutated DNA sequence of interest comprising:
(a) preparing a linear hemi-methylated double stranded plasmid vector DNA wherein the unmethylated strand (strand A) encodes a functional selectable marker gene and the methylated strand (strand B) has one or more insertions and/or deletions of four or more bases within the selectable marker gene of strand B relative to strand A and wherein the plasmid vector encodes a screenable marker gene;
(b) preparing a pool of double stranded DNAs containing a DNA sequence of interest using PCR overlap assembly from a pool of overlapping oligonucleotides wherein some of the resulting double stranded DNAs contain mutations, including insertions and deletions of various sizes, as well as base substitutions;
(c) denaturing and reannealing the double stranded DNAs to form heteroduplexes and homoduplexes;
(d) ligating the heteroduplexes and homoduplexes with the linear hemi-methylated double stranded plasmid vector to generate circular plasmid molecules;
(e) transforming the circular plasmid molecules into host cells having a DNA repair system;
(f) allowing time for the host cells to form colonies;
(g) scoring the colonies for the level of expression of the screenable marker;
(h) choosing colonies with a discernably different level of signal from the screenable marker than the level of signal observed with a control construct;
(i) isolating plasmid DNA from the chosen colonies; and
(j) evaluating the DNA sequence of the plasmids from the chosen colonies to detect plasmids with an unmutated DNA sequence of interest.

2. An in vitro method of isolating an unmutated DNA sequence of interest comprising:
(a) preparing a heteroduplex double stranded plasmid vector DNA wherein one strand (strand A) encodes a functional selectable marker gene and the second strand (strand B) encodes one or more insertions and/or deletions of four or more bases relative to strand A, with at least one occurring within the selectable marker gene of strand B, such that the selectable marker gene encoded by strand B is dysfunctional; and, wherein a screenable marker gene is also encoded by the vector DNA;
(b) preparing a target DNA pool of double stranded DNAs containing a DNA sequence of interest wherein at least some double stranded DNAs within the pool contain mutations;
(c) denaturing and reannealing the pool of double stranded DNAs of step (b) to form a DNA pool containing a mixture of heteroduplexes and homoduplexes;
(d) ligating the mixture of heteroduplexes and homoduplexes of step (c) with the double stranded heteroduplex plasmid vector DNA of step (a) to generate circular plasmid molecules;
(e) introducing the circular plasmid molecules of step (d) into cells having a functional DNA repair system;
(f) plating the cells resulting from step (e) onto solid medium and allowing time for the host cells to form colonies;
(g) scoring the colonies for the level of expression of the screenable marker gene of step (a);
(h) choosing colonies with a higher level of signal from the screenable marker compared to the level of signal observed with a control plasmid molecule in which both strands of the selectable marker gene correspond only to strand A of step (a);
i) isolating plasmid DNA from the chosen colonies; and
j) evaluating the DNA sequence of the plasmids from the chosen colonies to detect plasmids with an unmutated DNA sequence of interest.

3. The method of claim 2 wherein strand A of step (a) does not contain methylated adenine residues at GATC sequence sites, and strand B of step (a) does contain methylated adenine residues at GATC sequence sites.
